(19) United States
(12) Patent Application Publication
(10) Pub. No.: US 2006/0100214 A1
(43) Pub. Date: May 11, 2006

(54) Fast dissolving and taste masked oral dosage form comprising sildenafil

(76) Inventors: Wei Tian, Minety, Wiltshire (GB); John Langridge, Sint Oedenrode (NL)

Correspondence Address: MCANDREWS HELD & MALLOY, LTD, 500 WEST MADISON STREET, SUITE 3400, CHICAGO, IL, 60661, US

(21) Appl. No.: 10/525,680

(22) PCT Filed: Aug. 20, 2003
(86) PCT No.: PCT/GB03/03636

§ 371 Date: Sep. 19, 2005

(30) Foreign Application Priority Data
Aug. 21, 2002 (GB) 0219516.2

(51) Int. Cl. A61K 31/519; A61K 9/20

(52) U.S. Cl. 514/252.16; 424/464

(57) ABSTRACT

A fast dissolving and taste masked sildenafil solid dosage form comprising: (i) sildenafil granules which granules comprise at least 45% by weight of a salt of sildenafil, a solubilisation inhibitor for said salt of sildenafil and optionally a sweetening agent and (ii) one or more disintegrants wherein the disintegrants or combination of disintegrants are present in the form of agglomerates having an average agglomerated particle size of at least 50 μm, said agglomerates comprising at least 10% by weight of disintegrant.

The present invention relates to a fast dissolving and taste masked dosage form comprising a salt of sildenafil.

Fast disintegrating solid dosage forms for oral administration are known. Therapeutically, these dosage forms can lead to a rapid release of active component, thereby facilitating the increased absorption of certain active ingredients.

Freeze drying processes have been used to prepare fast disintegrating dosage forms. Depending on the manufacturing process, the product obtained is characterised by a highly porous microstructure of the soluble supporting agent (i.e. mannitol, glycine, lactose, gelatins etc.) in which the active is homogeneously dispersed. Although this technology produces a product which rapidly disintegrates in water or in the oral cavity, a drawback is represented by the poor physical integrity of its physical structure which severely limits further manufacturing operations such as forming blister packs.

Another significant drawback of the freeze drying technology in manufacturing such dosage forms is the high production costs because of the lengthy duration of each freeze drying cycle (normally from 24 to 48 hours). The complexity of the industrial plants is another important factor which prejudices the large scale use of this technology for the development of rapid disintegrating tablets. Moreover, the thermal shocks, as a direct consequence of each freeze drying cycle, might physically modify the physical-chemical properties of the outer membrane of microencapsulated particles.

Fast disintegrating tablets are known.

WO 99/47126 discloses a physiologically acceptable tablet comprising a compressed tablet formulation free of organic solvent residue that rapidly disintegrates when placed in a body cavity, comprising at least one water soluble non-saccharide polymer, the tablet has a crushing strength of between 0.5 kiloponds and 12.0 kiloponds.

U.S. Pat. No. 5,576,014 discloses intrabucally dissolving compressed mouldings comprising a saccharide having low mouldability having been granulated with a saccharide having high mouldability. The mouldings exhibit quick disintegration and dissolution in the buccal cavity and have an adequate hardness.

U.S. Pat. No. 6,024,981 discloses a hard, compressed, rapidly dissolvable dosage form adapted for direct oral dosing comprising an active ingredient and a matrix including a non-direct compression filler and a lubricant, the dosage form being adapted to rapidly dissolve in the mouth of a patient and thereby liberate the active ingredient.

WO 91/04757 discloses a solid dosage form adapted for direct oral administration to a human patient comprising an effective amount of at least one systemically distributable ingredient in the form of a tablet of a size and shape adapted for direct oral administration to a human patient, comprising at least one water or saliva activated effervescent disintegration agent, the tablet being substantially completely disintegrable upon exposure to water or saliva, and said at least one effervescent disintegration agent being present in an amount which is effective to aid in rapid disintegration of the tablet and to provide a distinct sensation of effervescence upon disintegration of the tablet in the mouth of a human patient.

U.S. Pat. No. 4,886,669 discloses a water-dispersible tablet comprising:
a) microparticles which contain at least one pharmaceutically active substance
b) at least one disintegrant and
c) a swellable material which is able to generate a high viscosity when coming into contact with water and which is selected from guar gum, xanthan gum, alginates, dextran, pectins, polysaccharides, sodium or calcium carboxymethylcellulose, hydroxypropylcellulose and hydroxypropylmethylcellulose,
   which tablet disintegrates rapidly in water forming a homogeneous suspension of high viscosity that can easily be swallowed.

WO99/44580 discloses a formulation for preparing a fast disintegrating tablet comprising a drug in multiparticulate form, one or more water insoluble excipients, one or more disintegrants; and optionally one or more substantially water soluble excipients, the amount of the ingredients being such as to provide a disintegration time for the tablet in the mouth in the order of seventy five seconds or less. It is stated superior tablet properties can be achieved by choosing appropriate amounts of the ingredients according to the classification shown below:
a) insoluble ingredient this includes the amount of drug either coated or uncoated and the amount of insoluble excipients including the insoluble inorganic salts used as filler diluents (e.g. di- or tri-basic calcium phosphate), organic filler (e.g. microcrystalline cellulose) or water insoluble lubricant (e.g. magnesium stearate, sodium steary fumarate, stearic acid or glyceryl behenate) and glidant (e.g. talc, silicone dioxide etc.)
b) substantially soluble components e.g. the amount of compression sugars (e.g. lactose), flavouring agents, sweeteners, binders and surfactants etc.
c) disintegrant, especially super-disintegrant, such as, maize starch or modified starches, cross-linked polyvinylpyrrolidone or sodium carboxymethylcellulose.

For constant ratios of ingredients a) and b) increasing the amount of disintegrant generally gives poorer friability values and increased disintegration times. In view of this the amount of super disintegrant c) should not be excessive and is therefore preferably in the range 0.5 to 30%, more preferably 1 to 20%, most preferably 2 to 15% by weight of tablet.

U.S. Pat. No. 6,106,861 discloses a rapidly disintergratable multiparticulate tablet which disintegrates in the mouth in less than forty seconds and which comprises an excipient and an active ingredient in the form of microcrystals coated with a coating agent. The excipient comprises, with respect to the mass of the tablet, from 3 to 15% by weight of at least one disintegration agent and from 40 to 90% by weight of at least one soluble diluent agent with binding properties consisting of a polyol having less than thirteen carbon atoms, said polyol being either in the directly compressible form which is composed of particles whose average diameter is from 100 to 500 micrometers or in the powder form which is composed of particles whose average diameter is less than 100 micrometers, said polyol being selected from the group consisting of mannitol, xylitol, sorbitol and maltitol, with the proviso that, when only one soluble diluent agent with binding properties is used, it is a polyol in the directly compressible form except sorbitol and, when at least two soluble diluent agents with binding properties are used, one is consisting of a polyol in the directly compressible form and the other is consisting of the same or another polyol in powder form, the proportion of directly compressible polyol to powder polyol being from 99/1 to 50/50.

WO00/09090 discloses an orally disintegrable tablet suitable for use in the delivery of at least one active ingredient in the form of microcapsules or powders characterised by between about 10 and about 80% of active ingredient containing microcapsules or powders by weight based on the weight of the tablet, said microcapsules or powder having a particle size ranging from between about 50 to 3,000 microns, an amount of at least one in-mouth viscosity enhancer, which is sufficient to provide a viscous, swallowable, organoleptically acceptable mass containing said microcapsules, within about three minutes when placed in a patients mouth, said in-mouth viscosity enhancer being selected from the group consisting of methylcellulose, hydroxypropylmethyl cellulose; hydroxyethyl cellulose, carbopol and silicon dioxide; optionally between 0 and 60% of a rapidly dissolvable sugar or sugar alcohol filler by weight of the tablet selected from the group consisting of sucrose, mannitol, xylitol, lactose and maltose; optionally between 0 and about 35% of a binder by weight of the tablet selected from the group consisting of microcrystalline cellulose and methyl cellulose; optionally between 0 and about 40% of a disintegrant by weight based on the weight of the tablet selected from the group consisting of sodium starch glycolate and crospovidone; and optionally between 0 and 50% of an effervescent couple based on the weight of the tablet.

EP-A-914818 discloses a tablet comprising sugar alcohol or saccharide having an averaging particle diameter of not more than 30 μm, an active ingredient, and a disintegrant. In a preferred embodiment a wet granulation method using purified water, ethanol or the like is used to prepare the tablets in the method, for example, granulation can be executed by means of a general granulator such as a fluid-bed granulator, a rotary stirring granulator or an extruding granulator. The granulated material is dried, and mixed with a lubricant, and thereafter compressed into predetermined shape. Binder, sour agent, foaming agent, sweetening agent, flavouring agent, or colourant can be added as additive.

EP 1145711 discloses a flash-melt pharmaceutical dosage form comprising a medicament and a combination of four excipients consisting of a superdisintegrant, a dispersing agent, a distributing agent and a binder. The four excipients may be dry granulated with the medicament and suitable conventional ingredients.

EP 0281200 discloses a pharmaceutical tablet comprising an amphoteric β-lactam antibiotic, and as disintegrants, a cellulose product and low-substituted hydroxypropylcellulose, in which the cellulose product is microcrystalline or microfine cellulose or a mixture of both. The tablet may be formed by compressing a granule of β-lactam antibiotic and microcrystalline cellulose and/or microfine cellulose.

WO01/39746 discloses a method for improving the compressibility of a superdisintegrant, comprising causing a partial or complete internal co-transformation of superdisintegrant particles, comprising temporarily opening up said particles and adding an augmenting agent which enhances the properties of the superdisintegrant relative to the unmodified particles of the superdisintegrant. The superdisintegrant may be mixed with an active agent and compressed into solid dosage forms or may be subjected to a wet granulation with the active ingredient.

The possible ways of combining superdisintegrants into tablets may be classified as follows:

(1) blend—where all components of the tablet are mixed and compressed to form the tablet;

(2) extragranulation—where components of the tablet other than the superdisintegrant are granulated and the granules mixed with the superdisintegrant and compressed to form the tablet;

(3) intragranulation—where all components of the tablet are granulated and the granules compressed to form a tablet and

(4) granular disintegrant—in accordance with the invention where the superdisintegrant is granulated separately from the active ingredient and preferably alone or in combination with a water-soluble filler, and the granules of superdisintegrant mixed with the other tablet components (which may be granular) and compressed to form a tablet.

Our co-pending International Application No. PCT GB2003/000844 discloses a fast disintegrating tablet comprising an active ingredient and one or more disintegrants characterised in that the tablet comprises agglomerates having an agglomerated particle size of at least 50 μm, said agglomerates comprising at least 10% by weight of a superdisintegrant selected from croscarmellose cellulose, crospovidone and sodium starch glycollate and being free of active ingredient.

The PCT applications further disclose a method of making a fast disintegrating tablet comprising the steps of

(i) forming agglomerates having an average agglomerated particle size of at least 50 μm and comprising one or more superdisintegrants selected from croscarmellose cellulose, crospovidone and sodium starch glycollate such that the agglomerates comprise at least 10% by weight of superdisintegrant and the agglomerates are free of active ingredient

(ii) mixing the agglomerates from step (i) with an active ingredient and optionally other tableting excipients, and

(iii) compressing the mixture from step (ii) to form a tablet.

One particular challenge facing the development of fast disintegration dosage form is the unpleasant taste of many drug actives. If not appropriately addressed, this can lead to serious problems of patient compliance. Particle coating technologies have been frequently used to mask the taste of the drug actives.

U.S. Pat. No. 6,106,881 discloses a rapidly disintegratable multiparticulate tablet which disintegrates in the mouth in less than forty seconds and which comprises an excipient and an active ingredient in the form of microcrystals coated with a coating agent.

U.S. Pat. No. 5,876,759 provides a compressed pharmaceutical dosage form, comprising:
a) at least one coated particle comprising at least one pharmaceutical coated with taste-masking coating comprising a blend of a first polymer selected from a cellulose acetate and cellulose acetate butyrate and a second polymer selected from polyvinyl pyrrolidone and hydroxypropyl cellulose, wherein the weight ratio of the first polymer to the second polymer is within the range of about 90:10 to about 50:50;
b) a water-disintegratable, compressible carbohydrate selected from mannitol, sorbitol, dextrose, xylitol, lactose and mixtures thereof, and
c) a binder selected from cellulose, polyvinyl pyrrolidone, starch, modified starch and mixtures thereof, the dosage form having a hardness of about 1.0 to 3.0 kp wherein the carbohydrate disintegrates in the oral cavity within 30 seconds after oral administration thereby allowing said coated particle to be swallowed.

It is well known among those skilled in the art that compression of the coated particles frequently leads to the fracture of coats, thereby causing the premature release of drug active within the oral cavity and leaving an unpleasant taste in the mouth. Accidental chewing of these particles can also cause the premature release of the active ingredient. Multiple coatings using the same or different coating compositions can be employed to minimise coat fracture, but may cause the unwanted problem of delayed release of active material.

Another disadvantage of incorporating coated particles into the fast dissolving dosage form is that the large coated particles can leave an unpleasant gritty mouthfeel within the oral cavity.

Other methods of taste masking besides particle coating are known.

WO 95/11671 discloses the use of an absorbate composition comprising magnesium aluminium silicate and two or more pharmaceutically acceptable actives in a fast dissolving dosage form.

EP 0582396 discloses a pharmaceutical composition having reduced bitterness relative to the bitterness of its constituent antibiotic agent, the composition comprising an azalide antibiotic, magnesium oxide (as an alkaline earth agent) and a pharmaceutically acceptable carrier.

The pharmaceutically active ingredients can be incorporated into fast dissolving dosage forms in the granular form.

U.S. Pat. No. 5,464,632 provides a method of making a granulate for use in the preparation of mouth-soluble, rapidly disintegrating tablets, which process comprises:
a) granulating in a fluid bed: (a) a polyalcohol; (b) an active ingredient; (c) from 1 to 30% of an edible acid wherein the edible acid is not part of an effervescent mixture consisting of an acid and a base; (d) optionally, other solid components selected from lubricants, sweetening agents, and flavours; and (e) an aqueous solution or aqueous dispersion of water soluble or water-dispersible polymer that provides 1-10% of the final weight of granulate; and
b) drying the granulate in the fluid bed.

WO 98/01114 provides a granulate, containing an active ingredient, having a solubility in water of 1>10, in admixture with a water dispersible cellulose, which is a microcrystalline cellulose and sodium carboxymethyl cellulose, in which the water dispersible cellulose is present in an amount of <15% wt %, the percentage based on the weight of active ingredient.

The incorporation of polymeric material in the granulation medium can also lead to the delayed release of the active ingredient.

Sildenafil citrate (1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine citrate) is a pharmaceutically active ingredient useful for the treatment of sexual dysfunction, such as, male erectile dysfunction (ED). ED can have a profound effect on the quality of life with subjects often reporting increased anxiety, loss of self-esteem, lack of self-confidence, tension and difficulty in the relationship with their partner. The prevalence of ED has been found to be associated with age. Complete ED has an estimated prevalence of 5% in men aged 40 years to 15% at age 70 years. The administration of sildenafil may benefit from the presentation of a fast disintegrating dosage form for both the geriatric patient group who may have swallowing difficulty and for the improved rate of drug absorption. However, sildenafil citrate has a very strong bitter taste, which renders the administration of fast dissolving dosage form of sildenafil an unpleasant experience.

WO 00/07596 discloses a pharmaceutical formulation which can be rapidly dissolved in water and which, as an active constituent, contains the phosphodiesterase (PDE) type 5 inhibitor sildenafil or the pharmaceutically safe salts thereof. There is no disclosure as to the mouthfeel and taste properties of the formulation.

WO 02/05820A1 provides solid dispersions of sildenafil citrate and certain highly water soluble sugars, which solid dispersions significantly increases the water solubility of sildenafil citrate. This requires a sophisticated process of preparing the solid dispersion.

US 20020002172A1 provides an orally disintegrating pharmaceutical preparation that comprises sildenafil free base together with a pharmaceutically acceptable carrier. The sildenafil free base is said to have very low water solubility and to be virtually taste free.

There is a need for an effective pharmaceutical dosage form incorporating a sildenafil salt that disintegrates rapidly within the oral cavity, has a pleasant mouthfeel without the bitter taste and does not require complicated manufacturing processes.

In accordance with the present invention there is provided a fast dissolving and taste masked sildenafil solid dosage form comprising:
i. sildenafil granules which granules comprise at least 45% by weight of a salt of sildenafil, a solubilisation inhibitor for said salt of sildenafil and optionally a sweetening agent and
ii. one or more disintegrants wherein the disintegrants or combination of disintegrants are present in the form of agglomerates having an average agglomerated particle size of at least 50 μm, said agglomerates comprising at least 10% by weight of disintegrant.
   The dosage form may additionally comprise one or more of water soluble fillers, diluents, lubricants, sweetening agents, flavouring agents and other auxiliary ingredients.

The dosage form of the invention rapidly disintegrates in the oral cavity within 30 seconds, preferably within 15 seconds. The dosage forms have a pleasing mouth feel and do not have the characteristic bitterness of sildenafil due to the presence of the solubilisation inhibitor. It has been found that in absence of the solubilisation inhibitor the characteristic bitterness of sildenafil cannot simply be masked by a sweetener alone.

The agglomeration of the disintegrant improves disintegration time in a simple and effective manner. Tablets made according to the invention may have a smooth surface, pleasing mouthfeel that is free of grittiness and disintegrate within thirty seconds, preferably within fifteen seconds according to the standard European Pharmacopoeia disintegration test.

Any pharmaceutically acceptable salt of sildenafil (1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine) can be used in the present invention, for example, hydrochloride, hydrobromide, sulphate or bisulphate, phosphate or hydrogen phosphate, acetate, citrate, fumarate, gluconate, lactate, maleate, succinate and tartrate. Sildenafil citrate is preferred.

The salt of sildenafil is generally present in an amount to provide from 5 to 150 mg/tablet, preferably 5 to 100 mg/tablet.

The sildenafil granules can be prepared by any means known in the art, for example, wet granulation, dry granulation, melt extrusion, extrusion-spheronisation, spray drying, co-spray drying, spray agglomeration etc.

The sildenafil granules contain at least 45% of a suitable salt of sildenafil. Preferably, the sildenafil granules contain at least 55% of a suitable salt of sildenafil. More preferably, the sildenafil granules contain more than 65% of a suitable salt of sildenafil granule.

The sildenafil granules contain a suitable agent that reduces the solubilisation of sildenafil salt. Sildenafil citrate has a solubility is 3.5 mg/ml at 23° C. in distilled water.

It is known that sildenafil citrate has a solubility profile depending on pH with the maximal solubility of approximately 24 mg/ml at pH2.0. Consequently, an effective method of reducing the solubility of sildenafil is through increased pH in the dissolution medium. Any pharmaceutically acceptable pH raising agent is acceptable. Suitable examples include sodium hydroxide, sodium carbonate, sodium bicarbonate, sodium phosphate, sodium citrate, calcium oxide, calcium carbonate, magnesium oxide and magnesium carbonate. The preferred pH raising agents are those with buffering capacity such as sodium carbonate, sodium bicarbonate and sodium phosphate.

Solubilisation inhibitors include those agents that will release the counterion of the sildenafil salt, for example sodium citrate for sildenafil citrate. Preferably, the solubilisation inhibitor is present at a sufficient amount as to form a saturated solution upon the disintegration of the tablet.

Other solubilisation inhibitors include those agents that can increase the hydrophobicity of the system, thereby reducing the water available to solubilise the sildenafil salt, for example, glyceryl monostearate, waxes, sodium stearyl lactate etc.

Optionally, a sweetening agent can be included in the sildenafil granules. Suitable sweetening agents include nutritive sweeteners such as sucrose, glucose, fructose, glucose, trehalose, galactose, mannitol, sorbitol, xylitol and intensive sweeteners such as aspartame, acesulfame K, sucrolose and NHDC.

It is preferred that all of the disintegrant is present in the form of agglomerates. However, disintegrant may be present in non-agglomerated form provided that at least 50%, preferably at least 75%, more preferably at least 90% by weight of disintegrant is agglomerated.

The agglomerates comprise at least 10%, preferably at least 25%, generally from 25 to 100% by weight disintegrant. The remainder of the agglomerates may comprise known tabletting ingredients including water-soluble and water insoluble fillers and/or diluents, active ingredient, binder, flavouring agents etc. Preferably the agglomerates comprise from 25 to 100% by weight disintegrant, the remainder being a water-soluble filler and optionally a binder, such as citric acid.

Disintegrating agents suitable for use in the present formulations include pharmaceutical excipients which facilitate the break-up of a tablet when it is placed in aqueous environment. Disintegrants once in contact with water, swell, hydrate, change in volume or form to produce a disruptive force that opposes the efficiency of the binder(s) causing the compressed tablet to break apart. They belong to different morphological classes and possess different functionality properties. A non-limiting list of the different classes of disintegrants or mixtures thereof which can be used in the formulations of the present invention is given below:

(1) natural starches, such as maize starch, potato starch etc., directly compressible starches such as starch 1500, modified starches such as carboxymethylstarches and sodium starch glycolate which are available as PRIMOJEL® and EXPLOTAB® and EXPLOSOL.

(2) cross-linked polyvinylpyrrolidones, e.g. crospovidones available as e.g. POLYPLASDONE XL® and KOLLIDON XL®.

(3) modified celluloses such as cross-linked sodium carboxymethylcelluloses available as, e.g., AC-DI-SOL®, PRIMELLOSE®, PHARMACEL XL®, EXPLOCEL® and NYMCEL ZSX®.

(4) Alginic acid and sodium alginate.

(5) Microcrystalline cellulose, e.g. AVICEL®, PHARMACEL®, EMCOCELL® and VIVAPUR®.

(6) Methacrylic acid-divinylbenzene copolymer salts available as e.g. AMBERLITE® IRP-88.

The agglomerates used in the invention comprise a superdisintegrant selected from:

(1) sodium starch glycolate which is available as PRIMOJEL® and EXPLOTAB® and EXPLOSOL.

(2) crospovidones available as e.g. POLYPLASDONE XL® and KOLLIDON XL®.

(3) croscarmellose cellulose as, e.g., AC-DI-SOL®, PRIMELLOSE®, PHARMACEL XL®, EXPLOCEL® and NYMCEL ZSX®.

Substantially water-soluble components that may be used in the present invention include sugars or soluble fillers, e.g. lactose, sucrose, dextrose, mannitol, etc., flavouring agents, sweeteners e.g. aspartame, saccharine etc., pH adjusting agents e.g. fumaric acid, citric acid, sodium acetate etc., binders e.g. polyethylene glycols, soluble hydroxyalkylcellulose, polyvinylpyrrolidone, gelatins, natural gums etc., surfactants e.g. sorbitan esters, docusate sodium, sodium lauryl sulphate, cetrimide etc., soluble inorganic salts e.g. sodium carbonate, sodium bicarbonate, sodium chloride etc.

Substantially water insoluble inorganic excipients include for example, water insoluble fillers and/or diluents, e.g. salts such as dibasic calcium phosphate, calcium phosphate tribasic, calcium sulfate and dicalcium sulfate. advantageously the particle size of the water insoluble inorganic excipient is such that at least 35% of the particles are larger than 75 μm. Most preferably at least 80% of the particles are larger than 75 μm.

The amount of disintegrant is generally at least 2% by weight of the tablet and preferably at least 4% by weight; a useful range being 4 to 20% by weight. Increasing levels of disintegrant tend to give poorer friability values for the tablet.

The amount of water-soluble and water-insoluble materials may be selected over wide ranges, depending upon the desired properties of the tablet.

The agglomeration of the disintegrant may be accomplished by any means known in the art, for example, wet granulation, dry granulation, extrusion, spray drying, co-spray drying, spray agglomeration etc. The average particle size of the agglomerator is at least 50 μm. Increasing particle size decreases disintegration time. Particle size ranges of from 75 to 500 μm are useful. Larger particle sizes may adversely affect the appearance of the tablets. The agglomerates are free of the active ingredient.

Tablets according to the present invention can be manufactured by well known tableting procedures. In common tableting processes, the agglomerates and other materials are deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion. Various tableting methods, well known to those skilled in the art, are comprehensively discussed throughout Pharmaceutical Dosage Forms: Tablets, Second Edition, edited by Herbert A. Lieberman et al., Copyright 1989 by Marcel Dekker, Inc., as well as other well known texts.

The invention will be illustrated by the following Examples in which the following ingredients were used:

| | |
|---|---|
| Polyplasdone ® XL-10: | crospovidone having an average particle |
| | size of about 30 μm |
| Mannitol: | mannitol having an average particle size of |
| | about 60 μm |
| Explotab ®: | sodium starch glycolate having an average |
| | particle size about 40 μm |

All parts and percentages are by weight unless otherwise stated.

Test Procedure: Determination of Oral Disintegration Time

Disintegration of the tablet was carried out by placing one tablet on the tongue of the subject. The subject was instructed not to bite the tablet but allowed to move the tablet gently within the mouth. The disintegration time was determined as the time between the tablet was placed in the mouth and when the last noticeable granules were disintegrated.

### EXAMPLES

### Example 1 (Comparative)

Formulation of Sildenafil Tablet:

| Formulation component | mg/tablet |
|---|---|
| Sildenafil granules | 185.75 |
| Agglomerated disintegrant granules | 60.00 |
| Aspartame | 1.25 |
| Lemon flavour | 0.50 |
| Magnesium stearate | 2.50 |
| Total | 250.0 |

Sildenafil Granules were Prepared According to the Following Formulation:

| Formulation component | % |
|---|---|
| Sildenafil citrate | 37.81 |
| Mannitol SD200 | 45.19 |
| Vivastar (sodium starch glycolate) | 7.00 |
| Citric acid | 5.00 |
| Lactitol | 5.00 |
| Total | 100.00 |

To prepare the sildenafil granule, citric acid and lactitol were dissolved in water. Sildenafil citrate, mannitol SD200 and sodium starch glycolate were blended in a food processor for 10 minutes, the citric acid/lactitol solution was added and mixed in, and the resulting wet granules were dried in a tray drier at 50° C.

Agglomerated disintegrant granules were prepared according to the following formulation:

| Formulation component | % |
|---|---|
| Mannitol (M60) | 60.00 |
| Polyplasdone XL-10 | 25.00 |
| Citric acid | 7.50 |
| Lactitol | 7.50 |
| Total | 100.00 |

To prepare the agglomerated disintegrant granules, citric acid and lactitol were dissolved in delonised water, mannitol and polyplasdone were dry mixed for 10 minutes in a food mixer. The citric acid/lactitol solution was added to the dry mixture to form wet granules. The wet granules were dried in a forced air oven at 50° C. to achieve a moisture level of less than 2%. The dried granules were screened and the 75 to 250 micron size range was obtained.

Tableting: the sildenafil granules and agglomerated disintegrant granules were placed in a suitable container. Aspartame and lemon flavour were screened, added to the mixture and blended for 10 minutes. Magnesium stearate was screened, added to the mixture and blended for a further 2 minutes. Tablets were prepared using a Stoke B2 rotary press fitted with 16 stations of ⅜ inch (9.525 mm) normal concave tooling.

The tablets had an average weight of 252 mg and a mean crushing strength of 1.1 kp. The oral disintegration time was 28 seconds with a strong bitter taste which lingered in the mouth for more than 5 minutes.

### Example 2 (Comparative)

Tablets incorporating concentrated sildenafil granules

Formulation of Sildenafil Tablet:

| Formulation component | mg/tablet |
|---|---|
| Sildenafil granules | 76.77 |
| Mannitol granules | 107.73 |
| Agglomerated disintegrant granules | 60.00 |
| Aspartame | 2.00 |
| Lemon flavour | 1.00 |
| Magnesium stearate | 2.50 |
| Total | 250.0 |

Sildenafil Granules were Prepared According to the Following Formulation:

| Formulation component | % |
|---|---|
| Sildenafil citrate | 91.50 |
| Lemon flavour | 1.00 |
| Aspartame | 2.50 |
| Citric acid | 2.50 |
| Lactitol | 2.50 |
| Total | 100.00 |

To prepare the sildenafil granule, citric acid and lactitol were dissolved in water. Sildenafil citrate, lemon flavour and aspartame were blended in a food processor for 10 minutes, the citric acid/lactitol solution was added and mixed in, and the resulting wet granules were dried in a tray drier at 50° C.

Mannitol granules were prepared according to the following formulation.

| Formulation component | % |
|---|---|
| Mannitol (SD200) | 91.00 |
| Vivastar (sodium starch glycolate) | 4.00 |
| Citric acid | 2.50 |
| Lactitol | 2.50 |
| Total | 100.00 |

To prepare the mannitol granules, citric acid and lactitol were dissolved in deionised water, mannitol and Vivastar were mixed for 10 minutes in a food mixer. The citric acid/lactitol solution was added to the dry mixture to form wet granules. The wet granules were dried in a forced air oven at 50° C. to achieve a moisture level of less than 1%.

Agglomerated disintegrant granules were prepared according to Example 1

Tableting: the sildenafil granules, mannitol granules, agglomerated disintegrant granules were placed in a suitable container. Aspartame and lemon flavour were screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed).

The tablets had an average weight of 252.5 mg and a mean crushing strength of 1.1 kp. The oral disintegration time was 12 seconds demonstrating the significant improvement in oral disintegration time when concentrated sildenafil granules were incorporated. The tablets had a strong bitter taste which lingered in the mouth for more than 5 minutes.

### Example 3 (Comparative)

Tablets incorporating concentrated sildenafil granules and an increased amount of sweetener

Formulation of Sildenafil Tablet:

| Formulation component | mg/tablet |
|---|---|
| Sildenafil granules | 90.00 |
| Mannitol granules | 95.00 |
| Agglomerated disintegrant granules | 60.00 |
| Lemon flavour | 2.50 |
| Magnesium stearate | 2.50 |
| Total | 250.0 |

Sildenafil Granules were Prepared According to the Following Formulation:

| Formulation component | % |
|---|---|
| Sildenafil citrate | 78.04 |
| Acesulfame K (high intensity | 16.40 |
| sweetener) | |
| Citric acid | 2.78 |
| Lactitol | 2.78 |
| Total | 100.00 |

To prepare the sildenafil granules, citric acid and lactitol were dissolved in water. Sildenafil citrate and acesulfame K were blended in a food processor for 10 minutes, the citric acid/lactitol solution was added and mixed in, and the resulting wet granules were dried in a tray drier at 50° C.

Mannitol granules were prepared according to Example 2.

Agglomerated disintegrant granules were prepared according to Example 1.

Tableting: the sildenafil granules, mannitol granules, agglomerated disintegrant granules were placed in a suitable container. Lemon flavour was screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed).

The tablets had an average weight of 251.1 mg and a mean crushing strength of 1.4 kp. The oral disintegration time was 15 seconds demonstrating the significant improvement in oral disintegration time when concentrated sildenafil granules were incorporated. The tablets had a strong bitter taste which lingered in the mouth for more than 5 minutes suggesting that the bitter taste can not be successfully masked by sweetener alone.

### Example 4 (Invention)

Tablets incorporating concentrated sildenafil granules and a solubilisation inhibitor

Formulation of Sildenafil Tablet:

| Formulation component | mg/tablet |
|---|---|
| Sildenafil granules | 110.20 |
| Mannitol granules | 62.50 |
| Agglomerated disintegrant granules | 60.00 |
| Lemon flavour | 5.00 |
| Acesulfame K | 9.80 |
| Magnesium stearate | 2.50 |
| Total | 250.0 |

Sildenafil Granules were Prepared According to the Following Formulation:

| Formulation component | % |
|---|---|
| Sildenafil citrate | 60.50 |
| Acesulfame K | 8.30 |
| Sodium carbonate | 26.20 |
| Citric acid | 2.50 |
| Lactitol | 2.50 |
| Total | 100.00 |

To prepare the sildenafil granules, citric acid and lactitol were dissolved in distilled water. Sildenafil citrate, sodium carbonate and acesulfame K were blended in a food processor for 10 minutes, the citric acid/lactitol solution was added and mixed in, and the resulting wet granules were dried in a tray drier at 50° C.

Mannitol granules were prepared according to Example 2.

Agglomerated disintegrant granules were prepared according to Example 1.

Tableting: the sildenafil granules, mannitol granules, agglomerated disintegrant granules were placed in a suitable container. Acesulfame K and lemon flavour was screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed).

The tablets had a pleasant sweet taste without the characteristic bitterness of sildenafil demonstrating the taste masking function of sodium carbonate. It was of interest to note that no effervescence was detected within the mouth.

### Example 5 (Invention)

Tablets incorporating concentrated sildenafil granules and a solubilisation inhibitor

Formulation of Sildenafil Tablet:

| Formulation component | mg/tablet |
|---|---|
| Sildenafil granules | 116.00 |
| Mannitol granules | 58.50 |
| Agglomerated disintegrant granules | 60.00 |
| Lemon flavour | 5.00 |
| Acesulfame K | 8.00 |
| Magnesium stearate | 2.50 |
| Total | 250.0 |

Sildenafil Granules were Prepared According to the Following Formulation:

| Formulation component | % |
|---|---|
| Sildenafil citrate | 63.70 |
| Acesulfame K | 8.71 |
| Sodium carbonate | 27.59 |
| Total | 100.00 |

To prepare the sildenafil granule, sildenafil citrate, sodium carbonate and acesulfame K were blended in a food processor for 10 minutes, distilled water was added was added and mixed in, and the resulting wet granules were dried in a tray drier at 50° C.

Mannitol granules were prepared according to Example 2.

Agglomerated disintegrant granules were prepared according to Example 1.

Tableting: the sildenafil granules, mannitol granules, agglomerated disintegrant granules were placed in a suitable container. Acesulfame K and lemon flavour was screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed).

The tablets had an average weight of 260.0 mg and a mean hardness of 0.9 kp. The oral disintegration time was 10 seconds demonstrating the significant improvement in oral disintegration time when concentrated sildenafil granules were incorporated. The tablets had a pleasant sweet taste without the characteristic bitterness of sildenafil demonstrating the taste masking function of sodium carbonate. No effervescence was detected within the oral cavity.

1. A fast dissolving and taste masked sildenafil solid dosage form comprising:
(i) sildenafil granules which granules comprise at least 45% by weight of a salt of sildenafil, a solubilisation inhibitor for said salt of sildenafil and optionally a sweetening agent and
(ii) one or more disintegrants wherein the disintegrants or combination of disintegrants are present in the form of agglomerates having an average agglomerated particle size of at least 50 μm, said agglomerates comprising at least 10% by weight of disintegrant.

2. A fast disintegrating solid dosage form as claimed claim 1 in which the sildenafil granules comprise at least 55% by weight of a salt of sildenafil.

3. A fast disintegrating solid dosage form as claimed in claim 2 in which the sildenafil granules comprise at least 65% by weight of a salt of sildenafil.

4. A fast disintegrating solid dosage form as claimed in claim 1 in which the salt of sildenafil is selected from hydrochloride, hydrobromide, sulphate or bisulphate, phosphate or hydrogen phosphate, acetate, citrate, fumarate, gluconate, lactate, maleat, succinate and tartrate.

5. A fast disintegrating solid dosage form as claimed in claim 1 in which the solubilisation inhibitor increases the pH when the sildenafil granules are dissolved in aqueous medium.

6. A fast disintegrating solid dosage form as claimed in claim 5 in which the solubilisation inhibitor is selected from sodium hydroxide, sodium carbonate, sodium bicarbonate, sodium phosphate, sodium citrate, calcium oxide, calcium carbonate, magnesium oxide and magnesium carbonate.

7. A fast disintegrating solid dosage form as claimed in claim 1 in which the solubilisation inhibitor releases the counter ion of the sildenafil salt.

8. A fast disintegrating solid dosage form as claimed in claim 1 in which the solubilisation inhibitor increases the hydrophobicity of the tablet.

9. A fast disintegrating solid dosage form as claimed in claim 8 in which the solubilisation inhibitor is selected from glyceryl monostearate, waxes and sodium stearyl lactate.

10. A fast disintegrating solid dosage form as claimed in claim 1 in which the salt of sildenafil is present in an amount to provide from 5 to 150 mg/solid dosage form of sildenafil.

11. A fast disintegrating solid dosage form as claimed in claim 10 in which the salt of sildenafil is present in an amount to provide from 5 to 100 mg/solid dosage form of sildenafil.

12. A fast disintegrating solid dosage form as claimed in claim 1 in which the agglomerates comprise at least 25% by weight of disintegrant.

13. A fast disintegrating solid dosage form as claimed in claim 12 in which the agglomerates comprise from 25 to 100% by weight of disintegrant.

14. A fast disintegrating solid dosage form as claimed in claim 1 in which at least 50% of the disintegrant is present in the tablet is in the form of said agglomerates.

15. A fast disintegrating solid dosage form as claimed in claim 14 in which at least 75% by weight of the disintegrant is present in the form of said agglomerates.

16. A fast disintegrating solid dosage form as claimed in claim 15 in which at least 90% by weight of the disintegrant is present in the form of said agglomerates.

17. A fast disintegrating solid dosage form as claimed in claim 16 in which all of the disintegrant in the tablet is present in the form of agglomerates.

18. A fast disintegrating solid dosage form as claimed in claim 1 in which the average particle size of the agglomerates is in the range 75 to 500 μm.

19. A fast disintegrating solid dosage form as claimed in claim 1 in which the tablet comprises at least 2% by weight of disintegrant.

20. A fast disintegrating solid dosage form as claimed in claim 19 in which the tablet comprises from 4 to 20% by weight of disintegrant.

21. A fast disintegrating solid dosage form as claimed in claim 1 in which the disintegrant is selected from croscarmellose cellulose, crospovidone and sodium starch glycollate.

22. A fast disintegrating solid dosage form as claimed in claim 1 in which the tablet additionally comprises water-soluble fillers or diluents selected from lactose, sucrose, dextrose and mannitol.

23. A method of making a fast disintegrating solid dosage form comprising the steps of:
(i) forming granules comprising at least 45% by weight of a salt of sildenafil, a solubilisation inhibitor for said salt of sildenafal and optionally a sweetening agent,
(ii) forming agglomerates having an average agglomerated particle size of at least 50 μm and comprising one or more disintegrants such that the agglomerates comprise at least 10% by weight disintegrant,
(iii) mixing the agglomerates from step (ii) with the granules of steps (i) and optionally other tableting excipients, and
(iv) compressing the mixture from step (iv) to form a solid dosage form.

24. A method of making a fast disintegrating solid dosage form in which the granules are prepared by wet granulation, dry granulation, melt extrusion, extrusion-spheronisation, spray drying, co-spray drying or spray agglomeration.

25. A method as claimed in claim 24 in which the granules comprise at least 45% by weight of a salt of sildenafil, a solubilisation inhibitor for said salt of sildenafil and optionally a sweetening agent.
